(19) United States
(12) Patent Application Publication
(10) Pub. No.: US 2025/0161621 A1
(43) Pub. Date: May 22, 2025

(54) ZONED KNIT HEADGEAR

(71) Applicant: KONINKLIJKE PHILIPS N.V., EINDHOVEN (NL)

(72) Inventors: JONATHAN SAYER GRASHOW, CHESWICK, PA (US); SJOERD EBBERINK, AMSTERDAM (NL); ERIK BELE HOGLUND, AMSTERDAM (NL); LLOYD MICHAEL SPENCER, PITTSBURGH, PA (US); JASON McGINNITY, II, PITTSBURGH, PA (US); BRENDAN HUTCHIESON, PITTSBURGH, PA (US)

(21) Appl. No.: 18/950,717

(22) Filed: Nov. 18, 2024

Related U.S. Application Data
(60) Provisional application No. 63/600,772, filed on Nov. 20, 2023.

(51) Int. Cl. A61M 16/06

(52) U.S. Cl. CPC A61M 16/0683

(57) ABSTRACT

A unitary knit headgear component for coupling a patient interface device to a head of a user includes a first zone comprising a first knit structure, and a second zone comprising a second knit structure different than the first knit structure, wherein the headgear component including the first zone and the second zoned are part of a continuously knit unitary structure. Several suitable exemplary knit structures and yarn and/or yarn combination for the headgear component are described in detail herein.

### CROSS-REFERENCE TO RELATED APPLICATIONS

This patent application claims the priority benefit under 35 U.S.C. § 119(e) of U.S. Provisional Application No. 63/600,772, filed on Nov. 20, 2023, the contents of which are herein incorporated by reference.

### BACKGROUND OF THE INVENTION

### 1. Field of the Invention

The disclosed concept relates generally to respiratory patient interface devices, and, in particular, to a knit fabric headgear structured to wrap around the patient's head for supporting a respiratory patient interface device during use that is knit directly as one piece comprising zones with distinct knit structures to enhance functionality.

### 2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible cushion on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. Such patient interface devices may also employ other patient contacting components, such as forehead supports, cheek pads and chin pads. The patient interface device is connected to a gas delivery tube or conduit and interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

Most modern headgear is constructed by cutting milled fabric and joining cut pieces (e.g., by sewing or welding) to build the desired shape with the desired functional and aesthetic properties. Some newer headgear are constructed using a knitting process whereby the headgear is knit as a single piece directly into the desired shape (eliminating the need to cut shapes from larger pieces of material as well as the need to join multiple pieces together). These knit headgear comprise a uniform knit structure that has consistent mechanical properties.

Adherence and compliance to therapy, such as CPAP or other pressure support therapies, is growing to be an industry-wide issue. Factors such as comfort and ease of use a patient interface device can greatly affect a patient's adherence and compliance to therapy. Thus, easier to use, comfortable and simplified designs for patient interface devices, and in particular to headgear used to hold the patient interface devices in place, are becoming expectations for any product that seeks to compete.

### SUMMARY OF THE INVENTION

In one embodiment, the disclosed concept provides a unitary knit headgear component for coupling a patient interface device to a head of a user. The headgear component includes a first zone comprising a first knit structure, and a second zone comprising a second knit structure different than the first knit structure, wherein the headgear component including the first zone and the second zoned are part of a continuously knit unitary structure. Several suitable exemplary knit structures and yarn and/or yarn combination for the headgear component are described in detail herein.

In another embodiment, a method of making a headgear component for coupling a patient interface device to a head of a user is provided. The method includes continuously knitting a first zone comprising a first knit structure and a second zone comprising a second knit structure different than the first knit structure, wherein the headgear component including the first zone and the second zoned are part of a continuously knit unitary structure.

In an alternative aspect, the displaced concept provides a knit headgear component for coupling a patient interface device to a head of a user that includes a rear portion structured and configured to engage a rear of the user's head while the headgear component coupled to the head of the user, wherein the rear portion includes a tunnel portion, a plurality of strap portions coupled to and extending from the rear portion, wherein the rear portion and the strap portions comprise an interlock knit and are part of a continuously knit unitary structure, and a bottom strap member formed separately from the unitary structure, wherein the bottom strap member comprises a terry cloth knit.

### BRIEF DESCRIPTION OF THE DRAWINGS

A full understanding of the invention can be gained from the following description of the preferred embodiments when read in conjunction with the accompanying drawings in which:

FIG. 1 is a top plan view of a headgear component, showing an enlarged portion thereof, according to a first exemplary embodiment of the disclosed concept;

FIG. 2 is a top plan view of a headgear component, showing an enlarged portion thereof, according to a second exemplary embodiment of the disclosed concept;

FIG. 3 is a top plan view of a headgear component according to a third exemplary embodiment of the disclosed concept;

FIG. 4 is a top plan view of a headgear component according to a second exemplary embodiment of the disclosed concept; and

FIG. 5 is a top plan view of a headgear component according to an alternative aspect of the disclosed concept;

FIG. 6 is a schematic diagram of a patient interface device according to a further exemplary embodiment of the disclosed concept;

FIG. 7 is a schematic diagram, in exploded form, of a headgear component forming part of the patient interface device of FIG. 6; and

FIG. 8 is a schematic diagram of a strap member according to a particular exemplary embodiment of the disclosed concept.

### DETAILED DESCRIPTION OF THE EXEMPLARY EMBODIMENTS

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body.

As used herein, the term “number” shall mean one or an integer greater than one (i.e., a plurality).

As used herein, the term “double knit” shall mean a fabric made with two different sets of needles such that the fabric is twice as thick as a single knit fabric and has two layers of knitting brought together by stitches that interlock with one another.

As used herein, the term “interlock knit” shall mean a compound fabric made by interlocking two single yarn ribbed knits together using two needles that continually cross over each other.

As used herein, the term “mesh knit” shall mean an open structure fabric comprised of regularly and/or irregularly spaced openings that create a breathable effect in the fabric. One example of a mesh knot is a pointelle, what has openings in the shape of chevrons.

As used herein, the term “ribbed knit” shall mean a fabric with distinct and visible patterns of vertical or horizontal ribs on its front and back.

As used herein, the term “terry cloth knit” shall mean a fabric made with multiple protruding loops of thread suitable for absorbing large amounts of liquid, such as water or sweat.

As used herein, the term “single jersey knit” shall mean a weft knitted fabric having a flat loop structure on one side and a reverse loop structure on the other side.

As used herein, the term “spacer knit” shall mean a fabric having two knit faces (top and a bottom) and yarns (e.g., without limitation microfilament yarns or conventional yarns) passing between the two faces to create a thicker “padding” type of 3D structure, sometimes referred to as a “spacer mesh.”

As used herein, the term “low stretch” shall mean a stiffness of 1000-5000 kPa (1500-2500 kPa in particular non-limiting exemplary embodiments).

As used herein, the term “high stretch” shall mean a stiffness of 50-1000 kPa (200-500 kPa in particular non-limiting exemplary embodiments).

As used herein, the term “higher friction” shall mean that a zone has a relatively higher surface roughness as compared to another zone of a knit material.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

This disclosed concept builds on the prior art by harnessing the advantages of knitting for headgear (less wasted material, no need for joining multiple pieces) while also providing a headgear with distinct zones, each with unique mechanical properties, to provide a headgear with superior comfort and ease of use. More specifically, the disclosed concept provides a headgear for use with patient interface devices that is made as a homogeneous, unitary structure formed by knitting the headgear as a single piece and providing within the headgear a plurality of different zones that have different mechanical structures. The different mechanical structures in the zones are result of using different knitting structures and/or different yarns and/or yarn combinations. Such a headgear may be formed by utilizing recently developed advanced/smart knitting technologies and machines, such as computer-controlled flatbed knitting machines, that are able to create different structures during a continuous knitting process. Such additional structures are formed by utilizing the machine to change the knit structure and/or yarns and/or yarn combinations “on-the-fly” during the automated knitting process. As a result, the headgear will be provided with different zones having different mechanical structures by way of continuous knitting. The disclosed concept is thus advantageous over the prior art, wherein headgear is formed with seams, edges and/or additional layers and/or by cutting and combining different fabrics in a multiple step process.

The disclosed concept contemplates that any of a number of different knit structures may be employed in the zones of the headgear, including, without limitation, a double knit (such as an interlock knit, a bird's eye knit or a Milano rib knit), a mesh knit, a ribbed knit, a single knit, a spacer knit, a terry cloth knit, a knit with disconnected layers (e.g., with tunnels or pockets), a single jersey knit, or a pique knit. In addition, the headgear with multiple zones as described herein according to the disclosed concept may be made using any of a number of different knitting techniques and/or processes, including, without limitation, flat bed knitting, circular knitting, webbing, weaving, or additive textile manufacturing. Furthermore, each zone may be formed using any of a number of different yarns or filaments and/or yarn/filament combinations and/or ratios of combinations, including, without limitation, monofilaments, filament fibers, staple fibers, core spun yarns (such as core spun spandex), textured yarns, natural and man-made fibers, such as cotton, cellulose fibers (semi synthetic), nylon, polyester, or cotton polyester, bio-based synthetic yarns, such as polymers derived from the castor bean, nylon-wrapped spandex, nylon+Lycra®, 10% Lycra®, or 20% Lycra®. In one exemplary embodiment, each zone may be made with the same yarn and/or yarn combinations, but with differing knit structures.

The yarn and/or yarn combinations for any zone may be chosen based on the properties thereof, due to yarn content, additives, or structure, such as, without limitation, being soft to touch, being cool to touch, being durable, having thermal regulation capability (e.g., being sweat/moisture absorbing), having quick drying capability (during use or after washing), having anti-odor capability, or having anti-bacterial capability. With respect to being soft to touch, the ultimate desire is to achieve a textile that feels smooth and soft, looks refined and provokes a feeling of comfort. This is of course a tradeoff between knit texture and yarn. With respect to durability of any yarn or yarn combination, it should not compromise the overall durability of the headgear, because if the headgear starts pilling or degrading quickly, the overall (perceived) quality will be impacted negatively. This is of course also a tradeoff between knit texture and yarn.

In addition, particular zones may have low stretch or high stretch properties. Low stretch is desirable for providing strap stability and overall fit of the headgear. If a headgear has too much stretch, the seal of the patient interface device (i.e., the cushion thereof) could leak and/or the mask of the patient interface device could lift off due to air pressure in the cushion. By reducing the stretch to a minimum, but ensuring the knitted texture still feels nice and comfortable, the headgear can make sure the fit of the mask is good and the seal of the cushion is kept during therapy.

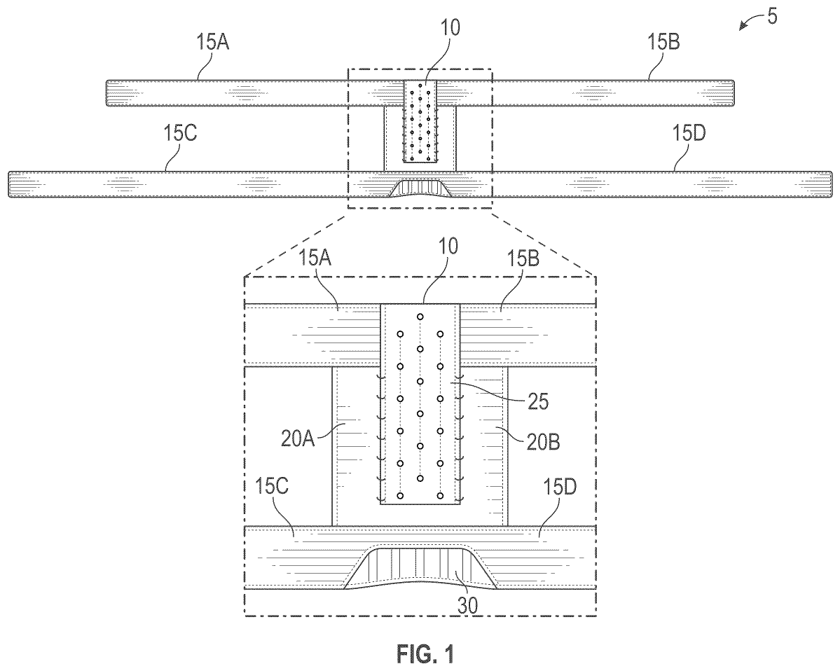

FIG. 1 is a top plan view of a headgear component 5, showing an enlarged portion thereof, according to a first non-limiting exemplary embodiment of the disclosed concept. Headgear component 5 is continuously knit as one piece (using a number of different knit structures and/or a number of different yarns and/or yarn combinations as described herein) such that headgear component 5 is a unitary component. Headgear component 5 includes a rear portion 10 structured and configured to engage the rear of a patient's head while headgear component 5 is used to secure a patient interface device, such as nasal/oral face mask, to the patient's head. A plurality of strap portions 15 are coupled to and extend from rear portion 10. Strap portions 15 are structured and configured to couple headgear component 5 to the patient interface device. As seen in FIG. 1, strap portions 15 include a top right strap portion 15A, a top left strap portion 15B, a bottom right strap portion 15C and a bottom left strap portion 15D. Each strap portion 15A-15D is structured to be adjustably coupled to a respective portion of the patient interface device by being attached to and/or inserted through a suitable attachment member, such as a loop member, provided as part of the patient interface device. In the exemplary embodiment, strap portions 15A-15D are provided with an adjustment mechanism, such as Velcro®, so that each strap portion 15 can be inserted through a respective attachment member comprising a loop member and folded back onto itself to selectively change the effective length of the strap member 15. Such selective adjustment capability allows for the strapping pressure provided by headgear component 5 to be adjustable while securing the patient interface device to the patient's head.

As seen in the enlarged portion of FIG. 1, rear portion 10 includes a plurality of zones, each of which provides different structure and functionality as described herein. Rear portion 10 includes a right lateral zone 20A and a left lateral zone 20B. As seen in FIG. 1, right lateral zone 20A and left lateral zone 20B are spaced from one another. In addition, right lateral zone 20A extends between a bottom edge of strap portion 15A and a top edge of strap portion 15C. Similarly, left lateral zone 20B extends between a bottom edge of strap portion 15B and a top edge of strap portion 15D. Both right lateral zone 20A and left lateral zone 20B are a ribbed knit for providing enhanced cushioning, fit and stability. Rear portion 10 further includes a middle zone 25.

As seen in FIG. 1, middle zone 25 is provided in the space that is present in between right lateral zone 20A and left lateral zone 20B. In addition, in the non-limiting exemplary embodiment, the top of middle zone 25 is provided in a space that exists between strap portion 15A and strap portion 15B, and the bottom of middle zone 25 extends to and is attached to the top edges of strap portion 15C and strap portion 15D at the junction of these two components. Middle zone 25 is a mesh knit for providing enhanced heat and moisture dissipation. Furthermore, as seen in FIG. 1, rear portion 10 further includes a bottom zone 30 that is attached to the bottom edges of strap portion 15C and strap portion 15D at the junction of these two components. In the exemplary embodiment, those bottom edges at the junction form an arc shape. As a result, in the exemplary embodiment, bottom zone 30 has an arc-shaped top edge and a straight bottom edge. Bottom zone 30 is a high stretch ribbed knit for providing enhanced stretch capability to, for example, alleviate neck discomfort. Finally, in the exemplary embodiment, at least the parts of strap portion 15A-15D that are immediately adjacent and coupled to rear portion 10 are a low stretch interlock knit for enhanced strap stability and ease of use.

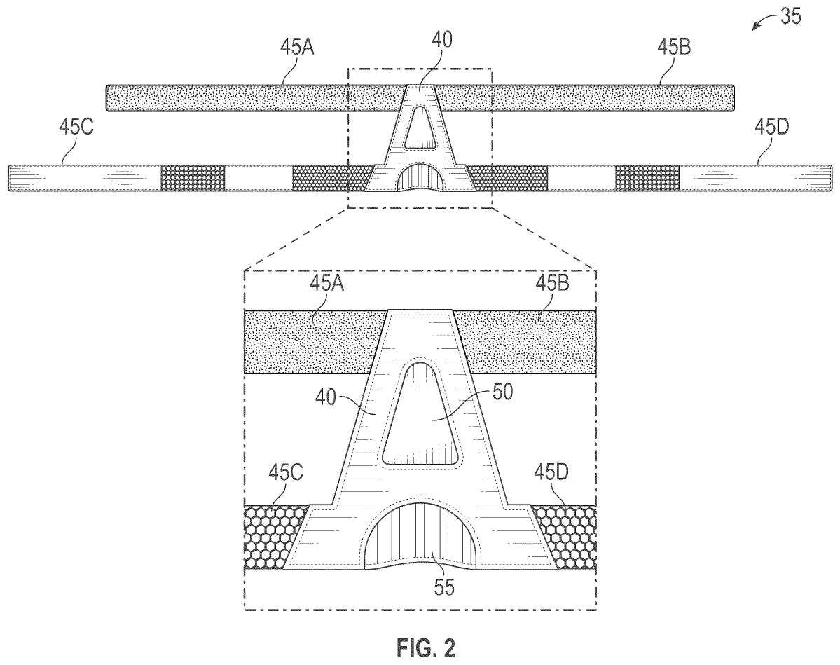

FIG. 2 is a top plan view of a headgear component 35, showing an enlarged portion thereof, according to a second non-limiting exemplary embodiment of the disclosed concept. Headgear component 35 is continuously knit as one piece (using a number of different knit structures and/or a number of different yarns and/or yarn combinations as described herein) such that headgear component 35 is a unitary component. Headgear component 35 includes a rear portion 40 structured and configured to engage the rear of a patient's head while headgear component 35 is used to secure a patient interface device, such as nasal/oral face mask, to the patient's head. A plurality of strap portions 45 are coupled to and extend from rear portion 40. Strap portions 45 are structured and configured to couple headgear component 35 to the patient interface device as described elsewhere herein. As seen in FIG. 2, strap portions 45 include a top right strap portion 45A, a top left strap portion 45B, a bottom right strap portion 45C and a bottom left strap portion 45D. Each strap portion 15A-15D is structured to be adjustably coupled to a respective portion of the patient interface device as described herein (see FIG. 1).

As seen in the enlarged portion of FIG. 2, rear portion 40 includes is a relatively thinner top zone 50 having less weight (e.g., half the weight) of the strap portions 45. Top zone 50 may be a single jersey knit, an interlock knit or a Milano knit having the weight as described. The goal of top zone 50 is to provide a thinner, more breathable fabric, while also giving structure to the overall headgear. Thus, it does not need to be as tight, heavy or structured as the rest of headgear component 35. Top zone 50 is a different execution of a breathable zone like middle zone 25 of the embodiment of FIG. 1. Furthermore, as seen in FIG. 2, rear portion 40 further includes a bottom zone 55 that is attached to the bottom edge of top zone 50. Bottom zone 55 has an arc-shaped top edge and a straight bottom edge. Bottom zone 55 is a high stretch ribbed knit for providing enhanced stretch capability to, for example, alleviate neck discomfort. Finally, in the exemplary embodiment, at least the parts of strap portions 45A-45D that are immediately adjacent and coupled to rear portion 40 are a higher friction low stretch double knit, such as an interlock knit, a Milano knot or a bird's eye knit. The goal of strap portions 45A-45D is to obtain the right balance in texture that makes it easy to move/adjust portions of an adjustment system provided with headgear component 35, but keep them fixated when not touched.

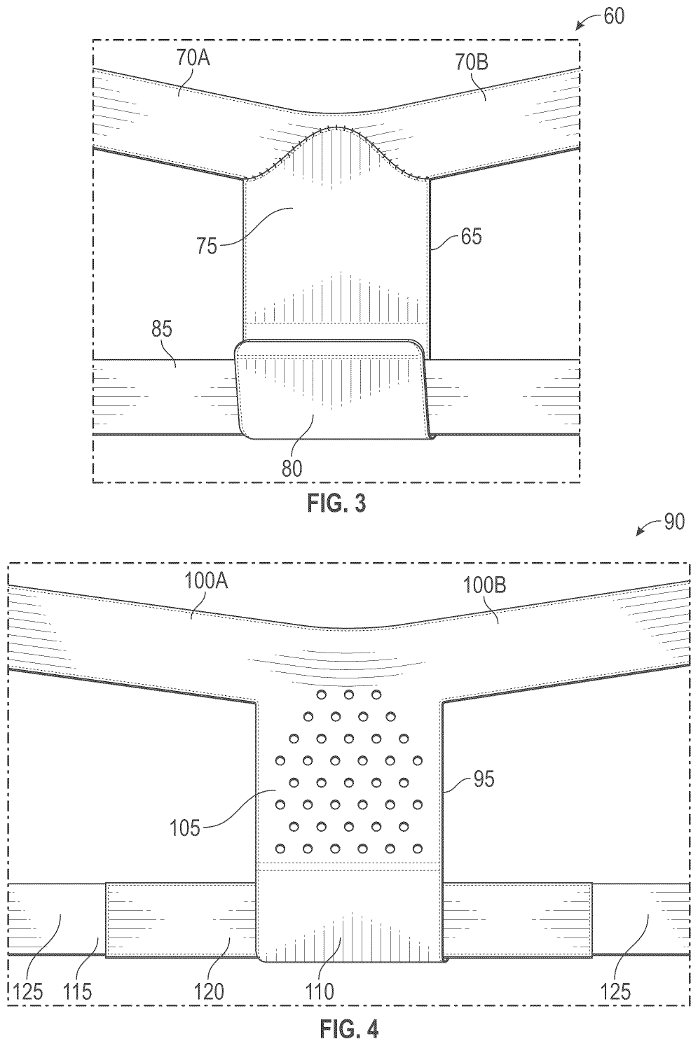

FIG. 3 is a top plan view of a headgear component 60 according to a third non-limiting exemplary embodiment of the disclosed concept. Headgear component 60 is continuously knit as one piece (using a number of different knit structures and/or a number of different yarns and/or yarn combinations as described herein) such that headgear component 60 is a unitary component. Headgear component 60 includes a rear portion 65 structured and configured to engage the rear of a patient's head while headgear component 60 is used to secure a patient interface device, such as nasal/oral face mask, to the patient's head. A plurality of interlock knit strap portions 70 are coupled to and extend from the top of rear portion 65. Strap portions 70 are structured and configured to couple headgear component 60 to the patient interface device as described elsewhere herein.

As seen in FIG. 3, strap portions 70 include a top right strap portion 70A and a top left strap portion 70B. Rear portion 65 includes a padded zone 75 made of a spacer knit to provide padding for improved comfort and moisture absorption. Rear portion 65 also includes a tunnel portion 80 provided at the bottom end thereof. Tunnel portion 80 may be constructed as a tubular knit or as a flat knit structure folded over on itself and sewn or otherwise attached to rear portion 65 to form the tunnel structure, with the latter being shown in FIG. 3 for illustrative purposes. Tunnel portion 80 is structured to receive therein and there-through a separately formed bottom strap member 85 made of an interlock stitch.

FIG. 4 is a top plan view of a headgear component 90 according to a fourth non-limiting exemplary embodiment of the disclosed concept. Headgear component 90 is continuously knit as one piece (using a number of different knit structures and/or a number of different yarns and/or yarn combinations as described herein) such that headgear component 90 is a unitary component. Headgear component 90 includes a rear portion 95 structured and configured to engage the rear of a patient's head while headgear component 90 is used to secure a patient interface device, such as nasal/oral face mask, to the patient's head. A plurality of interlock knit strap portions 100 are coupled to and extend from the top of rear portion 95. Strap portions 100 are structured and configured to couple headgear component 90 to the patient interface device as described elsewhere herein.

As seen in FIG. 4 strap portions 100 include a top right strap portion 100A and a top left strap portion 100B. Rear portion 95 includes a zone 105 made of a mesh knit to provide improved comfort and breathability. Rear portion 95 also includes a tunnel portion 110 provided at the bottom end thereof. Tunnel portion 110 may be constructed as a tubular knit or as a flat knit structure folded over on itself and sewn or otherwise attached to rear portion 95 to form the tunnel structure, with the former being shown in FIG. 4 for illustrative purposes. Tunnel portion 110 is structured to receive therein and there-through a separately formed bottom strap member 115 having a spacer knit central portion 120 and interlock knit lateral portions 125.

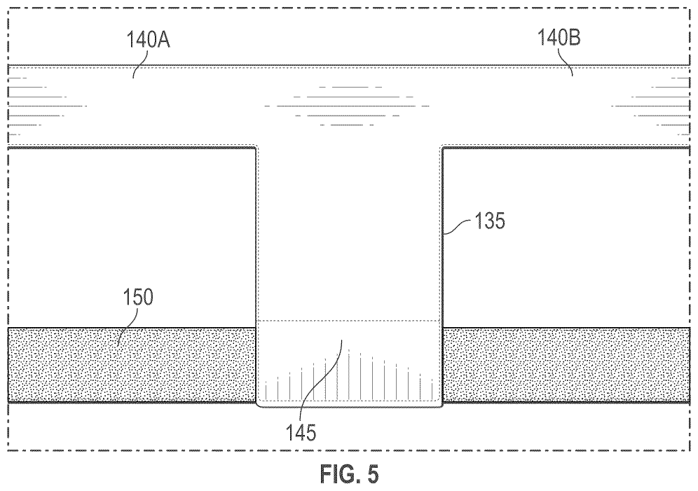

FIG. 5 is a top plan view of a headgear component 130 according to an aspect of the disclosed concept that is different than the previously described embodiments in that the different zones having different structures are not continuously knit as one piece, but instead are provided as two pieces that are coupled together. Headgear component 130 includes an interlock knit rear portion 135 structured and configured to engage the rear of a patient's head while headgear component 130 is used to secure a patient interface device, such as nasal/oral face mask, to the patient's head. A plurality of interlock knit strap portions 140 are coupled to and extend from the top of rear portion 135. Strap portions 140 are structured and configured to couple headgear component 130 to the patient interface device as described elsewhere herein. As seen in FIG. 5, strap portions 140 include a top right strap portion 140A and a top left strap portion 140B. Rear portion 135 includes a tunnel portion 145 provided at the bottom end thereof. Tunnel portion 145 may be constructed as a tubular knit or as a flat knit structure folded over on itself and sewn or otherwise attached to rear portion 135 to form the tunnel structure, with the former being shown in FIG. 5 for illustrative purposes. Tunnel portion 145 is structured to receive therein and there-through a separately formed terry cloth knit bottom strap member 150.

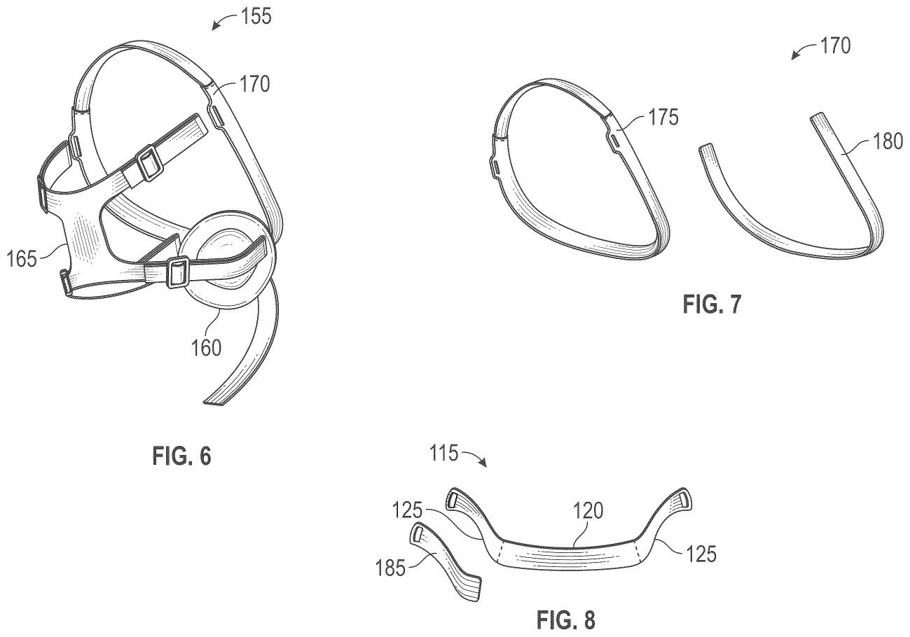

FIG. 6 is a schematic diagram of a patient interface device 155 according to a further exemplary embodiment of the disclosed concept. As seen in FIG. 6, patient interface device 155 includes a mask component 160 and a headgear apparatus comprising a first headgear component 165 and a second headgear component 170 which are both coupled to mask component 160. Headgear component 165 may be any of the headgear components 5, 35, 60, 90, or 130 as described herein, or any other suitable rear headgear device. Headgear component 170, shown in exploded form in FIG. 7, comprises a textile strap member 175 and a stiffening member 180 that is structured to be received within textile strap member 175. Textile strap member 175 is a headgear component that includes different zones as described herein (continuously knit as one piece using a number of different knit structures and/or a number of different yarns and/or yarn combinations), and, according to this embodiment, is of at least partly tubular construction. Textile strap member 175 is structured to receive therein (in the tubular parts thereof) stiffening member 180 in order to give stability and direction to patient interface device 155. In the exemplary embodiment, stiffening member 180 is a solid plastic part.

FIG. 8 is a schematic diagram of a bottom strap member 115 having a spacer knit central portion 120 and interlock knit lateral portions 125 according to one particular exemplary embodiment of the disclosed concept. In this particular embodiment, each of the lateral portions 125 is made of a tubular construction and is structured to receive there in a stiffening member 185 made of, for example, a plastic material in order to provide increased stability to a patient interface device.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A unitary knit headgear component for coupling a patient interface device to a head of a user, comprising:
a first zone comprising a first knit structure; and
a second zone comprising a second knit structure different than the first knit structure, wherein the headgear component including the first zone and the second zoned are part of a continuously knit unitary structure.

2. The unitary knit headgear component according to claim 1, wherein the first knit structure and the second knit structure is each one of a double knit comprising an interlock knit, a bird's eye knit or a Milano rib knit, a mesh knit, a ribbed knit, a single knit, a spacer knit, a terry cloth knit, a knit with disconnected layers, with tunnels or pockets, single jersey knit, or a pique knit.

3. The unitary knit headgear component according to claim 2, wherein the first knit structure and the second knit structure each comprises a yarn or yarn combination including a monofilament, a filament fiber, a staple fiber, a core spun yarns, a textured yarn, a natural fiber, a man-made fiber, bio-based synthetic yarn, a nylon-wrapped spandex yarn, a nylon+Lycra® yarn, 10% Lycra® yarn, or 20% Lycra® yarn.

4. The unitary knit headgear component according to claim 1, wherein the headgear component includes a rear portion structured and configured to engage a rear of the user's head while the headgear component coupled to the head of the user, and a plurality of strap portions coupled to and extending from the rear portion.

5. The unitary knit headgear component according to claim 4, wherein the first zone is part of the rear portion and the second zone is part of the plurality of strap portions.

6. The unitary knit headgear component according to claim 4, wherein the first zone and the second zone are both part of the rear portion.

7. The unitary knit headgear component according to claim 4, wherein the first zone and the second zone are both part of the plurality of strap portions.

8. The unitary knit headgear component according to claim 4, wherein the plurality of strap portions include a top right strap portion, a top left strap portion, a bottom right strap portion, and a bottom left strap portion, wherein the rear portion includes a right lateral zone and left lateral zone that are spaced from one another and middle zone provided in a space that is present in between the right lateral zone and the left lateral zone, wherein the right lateral zone extends between a bottom edge of the top right strap portion and a top edge of bottom right strap portion and the left lateral zone extends between a bottom edge of the top left strap portion and a top edge of the bottom left strap portion, wherein the first zone comprises the right lateral zone and the left lateral zone and wherein the first knit structure is a ribbed knit, and wherein the second zone is the middle zone and wherein the second knit structure is a mesh knit.

9. The unitary knit headgear component according to claim 8, wherein the rear portion includes a bottom zone positioned below the middle zone in between the bottom right strap portion and the bottom left strap portion, wherein the bottom zone comprises a third zone comprising a third knit structure, wherein the third knit structure is a high stretch ribbed knit.

10. The unitary knit headgear component according to claim 9, wherein the bottom zone has an arc-shaped top edge and a straight bottom edge.

11. The unitary knit headgear component according to claim 9, wherein the top right strap portion, the top left strap portion, the bottom right strap portion, and the bottom left strap portion comprise a fourth zone comprising a fourth knit structure, wherein the fourth knit structure is a low stretch double knit.

12. The unitary knit headgear component according to claim 11, wherein the low stretch double knit is a low stretch interlock knit.

13. The unitary knit headgear component according to claim 4, wherein the plurality of strap portions include a top right strap portion, a top left strap portion, a bottom right strap portion, and a bottom left strap portion, wherein the rear portion includes a top zone and a bottom zone positioned below the middle zone in between the bottom right strap portion and the bottom left strap portion, wherein the top zone is thinner than the plurality of strap portions, wherein the first zone comprises the top zone and wherein the first knit structure is a single jersey knit, an interlock knit or a Milano knit, and wherein the second zone is the bottom zone and wherein the second knit structure is a high stretch ribbed knit.

14. The unitary knit headgear component according to claim 13, wherein the bottom zone has an arc-shaped top edge and a straight bottom edge.

15. The unitary knit headgear component according to claim 13, wherein the top right strap portion, the top left strap portion, the bottom right strap portion, and the bottom left strap portion comprise a third zone comprising a third knit structure, wherein the third knit structure is a low stretch double knit.

16. The unitary knit headgear component according to claim 15, wherein the low stretch double knit is a low stretch interlock knit.

17. The unitary knit headgear component according to claim 4, wherein the plurality of strap portions include a top right strap portion and a top left strap portion, wherein the first zone is part of the rear portion and wherein the first knit structure is a spacer knit, and wherein the second zone is part of the top right strap portion and the top left strap portion and wherein the second knit structure is an interlock knit.

18. The unitary knit headgear component according to claim 17, wherein the rear portion includes a tunnel portion structured to receive therein and there-through a separately formed bottom strap member.

19. The unitary knit headgear component according to claim 4, wherein the plurality of strap portions include a top right strap portion and a top left strap portion, wherein the first zone is part of the rear portion and wherein the first knit structure is a mesh knit, and wherein the second zone is part of the top right strap portion and the top left strap portion and wherein the second knit structure is an interlock knit.

20. The unitary knit headgear component according to claim 1, further comprising a stiffening member, wherein the first zone includes a tunnel portion, and wherein the stiffening member is received within the tunnel portion.
